(19) United States
(12) Patent Application Publication
(10) Pub. No.: US 2008/0071348 A1
(43) Pub. Date: Mar. 20, 2008

(54) Medical Devices

(75) Inventors: Dennis A. Boismier, Shorewood, MN (US); Matthew Miller, Stillwater, MN (US); Michael Kuehling, Munich (DE)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/854,960

(22) Filed: Sep. 13, 2007

Related U.S. Application Data
(60) Provisional application No. 60/844,967, filed on Sep. 15, 2006.

(51) Int. Cl. A61F 2/06

(52) U.S. Cl. 623/1.15

(57) ABSTRACT

Medical devices, such as endoprostheses, and methods of making the devices are disclosed.

### CROSS-REFERENCE TO RELATED APPLICATIONS

This application claims priority under 35 USC §119(e) to U.S. patent application Ser. No. 60/844,967, filed on Sep. 15, 2006, the entire contents of which are hereby incorporated by reference.

### TECHNICAL FIELD

The invention relates to medical devices, such as, for example, endoprostheses, and methods of making the devices.

### BACKGROUND

The body includes various passageways, such as arteries, other blood vessels, and other body lumens. These passageways sometimes become occluded or weakened. For example, the passageways can be occluded by a tumor, restricted by plaque, or weakened by an aneurysm. When this occurs, a passageway can be reopened or reinforced, or even replaced, with a medical endoprosthesis. An endoprosthesis is typically a tubular member that is placed in a lumen in the body. Examples of endoprostheses include stents, stent-grafts, and covered stents.

An endoprosthesis can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded (e.g., elastically or through a material phase transition). During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

To support a passageway and keep the passageway open, endoprostheses are sometimes made of relatively strong materials, such as stainless steel or Nitinol (a nickel-titanium alloy), formed into struts or wires.

### SUMMARY

In one aspect, the invention features medical devices (e.g., endoprostheses) that include one or more metals (e.g., biocrodible metals) and/or foams (e.g., bioerodible foams), and methods of making the devices. In some embodiments, the medical devices can include bioerodible metal foams. The erosion of the medical devices can be controlled. For example, the medical devices may include pores of a particular size, location, and/or arrangement that are selected to result in a desired pattern and/or rate of erosion of the medical devices. In certain embodiments, the medical devices can include one or more therapeutic agents. In embodiments in which the medical devices include both therapeutic agents and bioerodible metals and/or foams, the therapeutic agents may be released from the medical devices as the bioerodible metals and/or foams erode.

In another aspect, the invention features an endoprosthesis (e.g., a stent) including a generally tubular member. The generally tubular member includes a bioerodible foam including a metal.

In an additional aspect, the invention features an endoprosthesis (e.g., a stent) including a generally tubular member including a biocrodible metal and having a first region including a least one hole and a second region that does not include any holes. Both the first region and the second region include the bioerodible metal.

In a further aspect, the invention features a method of making an endoprosthesis (e.g., a stent) including a generally tubular member. The method includes heating a powder including a bioerodible metal to form the generally tubular member.

In another aspect, the invention features a method of making an endoprosthesis (e.g., a stent) including a generally tubular member. The method includes treating a biocrodible foam including a metal to form the generally tubular member.

In an additional aspect, the invention features a method of making an endoprosthesis (e.g., a stent) including a generally tubular member. The method includes forming at least one hole in a first region of the generally tubular member so that the first region includes the hole and a second region of the generally tubular member does not include any holes. The generally tubular member includes a bioerodible metal.

Embodiments can include one or more of the following features.

The metal can be iron, magnesium, zinc, aluminum, or a combination thereof.

The generally tubular member can include a material that is non-bioerodible. The generally tubular member can include a polymer (e.g., a bioerodible polymer, a non-bioerodible polymer) and/or can include another metal (e.g., a bioerodible metal, a non-biocrodible metal). The bioerodible foam can include pores, and the polymer and/or the other metal can be disposed within the pores. The generally tubular member can include one or more metal oxides, ceramics, or combinations thereof.

The generally tubular member can include a connector and/or a band including at least one of the first region and the second region.

The bioerodible foam can include a pore having a dimension of at least about 20 nanometers (e.g., at least about 50 nanometers, at least about 100 nanometers, at least about 250 nanometers, at least about 500 nanometers, at least about 750 nanometers, at least about one micron, at least about five microns, at least about 10 microns, at least about 25 microns, at least about 40 microns, at least about 50 microns, at least about 75 microns) and/or at most about 100 microns (e.g., at most about 75 microns, at most about 50 microns, at most about 40 microns, at most about 25 microns, at most about 10 microns, at most about five microns, at most about one micron, at most about 750 nanometers, at most about 500 nanometers, at most about 250 nanometers, at most about 100 nanometers, at most about 50 nanometers). The bioerodible foam can include a pore having a dimension of from about 20 nanometers to about 10 microns, and another pore having a dimension of from about 10 microns to about 100 microns. The pores can occupy at least about five percent (e.g., at least about 10 percent, at least about 20 percent, at least about 30 percent, at least about 40 percent, at least about 50 percent, at least about 60 percent, at least about 70 percent, at least about 80 percent, at least about 90 percent), and/or at most about 95 percent (e.g., at most about 90 percent, at most about 80 percent, at most about 70 percent, at most about 60 percent, at most about 50 percent, at most about 40 percent, at most about 30 percent, at most about 20 percent, at most about 10 percent), of the volume of the biocrodible foam.

The second region can include the bioerodible metal. The generally tubular member can include a connector, a band, or a combination thereof, and the first and/or second region can be located in the connector, the band, or the combination thereof.

The endoprosthesis can include a therapeutic agent.

Heating a powder including a bioerodible metal can include exposing the powder to a temperature of at least about 400° C. The powder can include at least one particle having a dimension of at least about 20 nanometers (e.g., at least about 50 nanometers, at least about 100 nanometers, at least about 250 nanometers, at least about 500 nanometers, at least about 750 nanometers, at least about one micron, at least about five microns, at least about 10 microns, at least about 25 microns, at least about 40 microns, at least about 50 microns, at least about 75 microns) and/or at most about 100 microns (e.g., at most about 75 microns, at most about 50 microns, at most about 40 microns, at most about 25 microns, at most about 10 microns, at most about five microns, at most about one micron, at most about 750 nanometers, at most about 500 nanometers, at most about 250 nanometers, at most about 100 nanometers, at most about 50 nanometers).

Treating a bioerodible foam including a metal to form the generally tubular member can include molding the bioerodible foam to form the generally tubular member. The generally tubular member can include a generally tubular portion, and treating a bioerodible foam including a metal to form the generally tubular member can include coating the generally tubular portion with the biocrodible foam. The method can include combining the bioerodible foam with another metal. The bioerodible foam can include pores, and combining the foam with another metal can include infiltrating the pores with the other metal. The method can include combining the biocrodible foam with a polymer. The bioerodible foam can include pores, and combining the bioerodible foam with a polymer can include infiltrating the pores with the polymer. The polymer can include a therapeutic agent. The method can include adding a therapeutic agent to the generally tubular member.

Embodiments can include one or more of the following advantages.

In certain embodiments, a medical device (e.g., an endoprosthesis) including a bioerodible metal can be used to temporarily treat a subject without permanently remaining in the body of the subject. For example, the medical device may be used for a certain period of time (e.g., to support a lumen of a subject), and then may erode after that period of time is over.

In some embodiments, a medical device (e.g., an endoprosthesis) including a bioerodible metal can be relatively strong and/or can have relatively high structural integrity, while also having the ability to erode after being used at a target site.

In certain embodiments, a medical device (e.g., an endoprosthesis) can provide a controlled release of one or more therapeutic agents into the body of a subject. For example, in some embodiments in which a medical device includes a bioerodible metal and a therapeutic agent, the erosion of the bioerodible metal can result in the release of the therapeutic agent over a period of time.

In certain embodiments, a medical device (e.g., an endoprosthesis) can include a bioerodible metal having one or more pores and/or holes. The number, size, arrangement, and/or location of the pores and/or holes can be selected to provide a desired pattern and/or rate of erosion of the medical device. In some embodiments, the number, size, arrangement, and/or location of the pores and/or holes can be selected to result in the formation of relatively small erosion products that can be unlikely to have an adverse effect on the body.

In certain embodiments, a medical device (e.g., an endoprosthesis) can include a biocrodible material and at least one other material that is either bioerodible or non-bioerodible. The other material may, for example, enhance the strength and/or structural integrity of the medical device. In some embodiments, the other material can be a therapeutic agent, and as the bioerodible material of the medical device erodes, the therapeutic agent can be released (e.g., into a target site in a body of a subject). In certain embodiments, a medical device can include multiple (e.g., two, three) different bioerodible materials. The relative amounts of the biocrodible materials, and/or their locations in the medical device, can be selected to provide a desired pattern and/or rate of erosion of the medical device.

In some embodiments, the pores in a metal foam (e.g., a bioerodible metal foam) of a medical device (e.g., an endoprosthesis) can be used to store a therapeutic agent. In certain embodiments, the medical device can also be coated with a biocrodible material that erodes after the medical device has been delivered to a target site in the body of a subject, thereby allowing the therapeutic agent to elute from the pores.

In some embodiments in which a medical device (e.g., an endoprosthesis) includes both a biocrodible material and a therapeutic agent, the erosion rate of the bioerodible material can be independent of the elution rate of the therapeutic agent. As an example, in certain embodiments, a medical device can include a bioerodible foam. A biocrodible polymer including a therapeutic agent can be disposed within the pores of the foam. As the polymer erodes, it can release the therapeutic agent at a rate that is different from the erosion rate of the foam. In certain embodiments, the foam can erode before all of the therapeutic agent has been released from the polymer. The remaining polymer can continue to elute the therapeutic agent. The therapeutic agent can be selected, for example, to help alleviate the effects, if any, of the erosion of the foam on the body of the subject.

In some embodiments, a medical device (e.g., an endoprosthesis) including one or more metals (e.g., biocrodible metals) can be relatively radiopaque. This radiopacity can give the medical device enhanced visibility under X-ray fluoroscopy. Thus, the position of the medical device within the body of a subject may be able to be determined relatively easily.

An erodible or biocrodible endoprosthesis, e.g., a stent, refers to a device, or a portion thereof, that exhibits substantial mass or density reduction or chemical transformation, after it is introduced into a patient, e.g., a human patient. Mass reduction can occur by, e.g., dissolution of the material that forms the device and/or fragmenting of the device. Chemical transformation can include oxidation/reduction, hydrolysis, substitution, and/or addition reactions, or other chemical reactions of the material from which the device, or a portion thereof, is made. The erosion can be the result of a chemical and/or biological interaction of the device with the body environment, e.g., the body itself or body fluids, into which it is implanted and/or erosion can be triggered by applying a triggering influence, such as a chemical reactant or energy to the device, e.g., to increase a reaction rate. For example, a device, or a portion thereof, can be formed from an active metal, e.g., Mg or Ca or an alloy thereof, and which can erode by reaction with water, producing the corresponding metal oxide and hydrogen gas (a redox reaction). For example, a device, or a portion thereof, can be formed from an erodible or bioerodible polymer, or an alloy or blend erodible or bioerodible polymers which can erode by hydrolysis with water. The erosion occurs to a desirable extent in a time frame that can provide a therapeutic benefit. For example, in embodiments, the device exhibits substantial mass reduction after a period of time which a function of the device, such as support of the lumen wall or drug delivery is no longer needed or desirable. In particular embodiments, the device exhibits a mass reduction of about 10 percent or more, e.g. about 50 percent or more, after a period of implantation of one day or more, e.g. about 60 days or more, about 180 days or more, about 600 days or more, or 1000 days or less. In embodiments, the device exhibits fragmentation by erosion processes. The fragmentation occurs as, e.g., some regions of the device erode more rapidly than other regions. The faster eroding regions become weakened by more quickly eroding through the body of the endoprosthesis and fragment from the slower eroding regions. The faster eroding and slower eroding regions may be random or predefined. For example, faster eroding regions may be predefined by treating the regions to enhance chemical reactivity of the regions. Alternatively, regions may be treated to reduce erosion rates, e.g., by using coatings. In embodiments, only portions of the device exhibits erodibilty. For example, an exterior layer or coating may be erodible, while an interior layer or body is non-erodible. In embodiments, the endoprosthesis is formed from an erodible material dispersed within a non-erodible material such that after erosion, the device has increased porosity by erosion of the erodible material.

Erosion rates can be measured with a test device suspended in a stream of Ringer's solution flowing at a rate of 0.2 m/second. During testing, all surfaces of the test device can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

As used herein, a foam has a complex, reticulated structure having interstices, pores, cells, and/or passages that extend wholly or partially across the foam. The foam may have portions that have been fused to other portions, and/or portions that terminate without being fused to other portions. The foam typically includes a multitude of pathways and obstructions of the pathways such that there is no line of sight extending across the entire foam. In some embodiments, there is an interconnecting network of continuous and meandering pores or voids through the foam. The microscopic network structure of the foam can resemble the microscopic structure of a sponge, cancellous bone, slightly bonded felt, or three-dimensional layers of netting.

As used herein, an “alloy” means a substance composed of two or more metals or of a metal and a nonmetal intimately united, for example, by being fused together and dissolving in each other when molten.

Other aspects, features, and advantages are in the description, drawings, and claims.

### DESCRIPTION OF DRAWINGS

FIG. 1A is a perspective view of an embodiment of a stent in a compressed condition.

FIG. 1B is a perspective view of the stent of FIG. 1A, in an expanded condition.

FIG. 1C is a cross-sectional view of the stent of FIG. 1A, taken along line 1C-1C.

FIG. 2A is a perspective view of an embodiment of a stent.

FIG. 2B is a cross-sectional view of the stent of FIG. 2A, taken along line 2B-2B.

FIG. 3 is a cross-sectional view of an embodiment of a stent.

FIG. 4A is a perspective view of an embodiment of a stent.

FIG. 4B is a cross-sectional view of the stent of FIG. 4A, taken along line 4B-4B.

FIG. 5A is a perspective view of an embodiment of a stent.

FIG. 5B is an enlarged view of region 5B of the stent of FIG. 5A.

### DETAILED DESCRIPTION

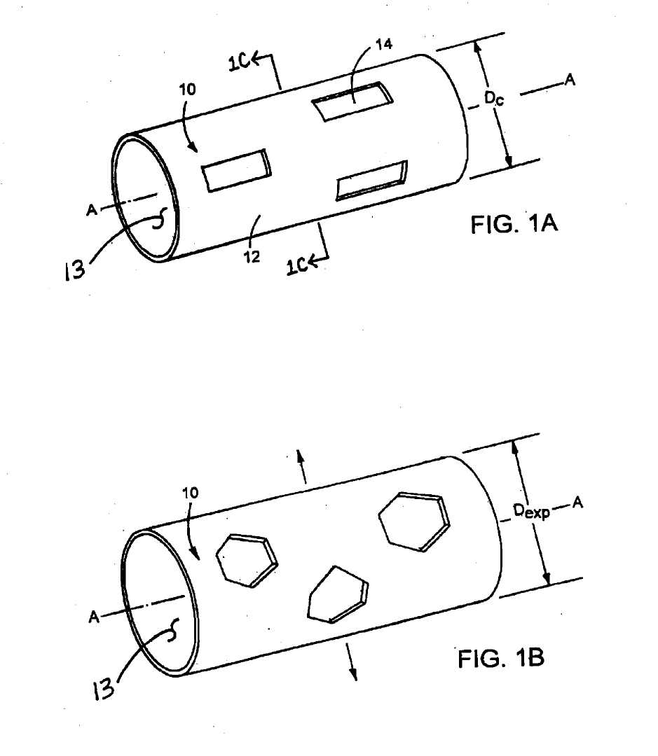

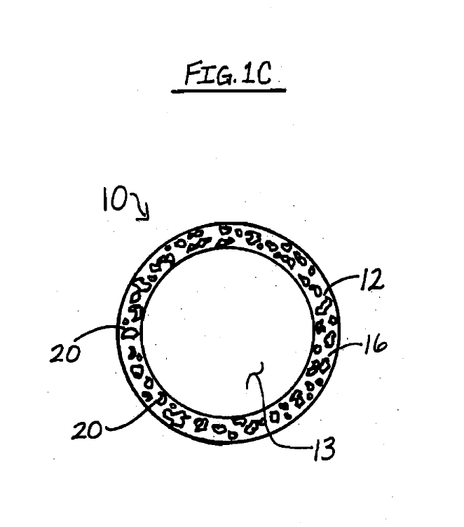

FIG. 1A shows a stent 10 including a generally tubular member 12 capable of supporting a body lumen and having a longitudinal axis A-A and defining a lumen 13. Generally tubular member 12 includes apertures 14 that are provided in a pattern to facilitate stent functions (e.g., radial expansion) and lateral flexibility. FIG. 1A shows stent 10 in a compressed condition, such that stent 10 has a relatively small diameter D_{c} suitable for delivery into a lumen of a subject. As shown in FIG. 1B, once stent 10 has been delivered into a lumen of a subject, stent 10 is expanded to a larger diameter, Dₑₓₚ. This larger diameter can allow stent 10 to contact the walls of the lumen. A stent such as stent 10 may be expanded by a mechanical expander (e.g., an inflatable balloon), or may be self-expanding.

FIG. 1C shows a cross-sectional view of stent 10. As shown in FIG. 1C, generally tubular member 12 includes (e.g., is formed of) a metal foam 16 including cells or pores 20. Pores 20 form an interconnected network, so that metal foam 16 is an open-cell foam. While pores 20 are shown as having an irregular cross-sectional shape, in some embodiments, the pores in a metal foam can have one or more other cross-sectional shapes. For example, a pore in a metal foam can be circular, oval (e.g., elliptical), and/or polygonal (e.g., triangular, square) in cross-section.

In some embodiments, metal foam 16 can be bioerodible, so that generally tubular member 12 also is bioerodible. In certain embodiments in which metal foam 16 is bioerodible, generally tubular member 12 of stent 10 can erode after stent 10 has been used at a target site. Because metal foam 16 is an open-cell foam, generally tubular member 12 may exhibit relatively uniform erosion.

Examples of bioerodible metals include alkali metals, alkaline earth metals (e.g., magnesium), iron, zinc, and aluminum. Metal foam 16 can include one metal, or can include multiple (e.g., two, three, four, five) metals. In some embodiments, metal foam 16 can include one or more metals that are in the form of metal alloys. Examples of bioerodible metal alloys include alkali metal alloys, alkaline earth metal alloys (e.g., magnesium alloys), iron alloys (e.g., alloys including iron and up to seven percent carbon), zinc alloys, and aluminum alloys. Bioerodible materials are described, for example, in Weber, U.S. Patent Application Publication No. US 2005/0261760 A1, published on Nov. 24, 2005, and entitled “Medical Devices and Methods of Making the Same”; Colen et al., U.S. Patent Application Publication No. US 2005/0192657 A1, published on Sep. 1, 2005, and entitled “Medical Devices”; Weber, U.S. patent application Ser. No. 11/327,149, filed on Jan. 5, 2006, and entitled “Biocrodible Endoprostheses and Methods of Making the Same”; Bolz, U.S. Pat. No. 6,287,332; Heublein, U.S. Patent Application Publication No. US 2002/0004060 A1, published on Jan. 10, 2002, and entitled “Metallic Implant Which is Degradable In Vivo”; and Park, Science and Technology of Advanced Materials, 2, 73-78 (2001).

In some embodiments, a medical device (e.g., stent 10) or a component of a medical device (e.g., generally tubular member 12) that is formed of one or more bioerodible materials can erode over a period of at least about five days (e.g., at least about seven days, at least about 14 days, at least about 21 days, at least about 28 days, at least about 30 days, at least about six weeks, at least about eight weeks, at least about 12 weeks, at least about 16 weeks, at least about 20 weeks, at least about six months, at least about 12 months). In some embodiments in which a medical device includes one or more radiopaque materials, the erosion of the medical device within the body of a subject can be monitored using X-ray fluoroscopy. In certain embodiments, the erosion of a medical device within the body of a subject can be monitored using intravascular ultrasound.

In certain embodiments, a medical device (e.g., a medical device including magnesium) can be designed to erode by a bulk erosion process, in which water and/or other body fluids penetrate the bioerodible material and cause it to erode in bulk. In some embodiments, a medical device (e.g., a medical device including magnesium and/or iron) can be designed to erode by a surface erosion process, in which water and/or other body fluids cause the medical device to erode at its surface. In certain embodiments, a medical device that erodes by a bulk erosion process can erode at a faster rate than a medical device that erodes by a surface erosion process. In some embodiments, a medical device that erodes by a surface erosion process may experience a relatively controlled erosion, and/or may be relatively unlikely to result in an inflammatory reaction by the body.

In certain embodiments, generally tubular member 12 can erode at a faster rate than a generally tubular member that does not include any pores, but is otherwise comparable to generally tubular member 12. Without wishing to be bound by theory, it is believed that pores 20 can cause a relatively large surface area of bioerodible metal to be exposed to blood and/or other body fluids at a target site. As a result, generally tubular member 12 may erode at a faster rate than a generally tubular member that does not include any pores, or that includes fewer pores than generally tubular member 12.

In some embodiments, stent 10 can include one or more therapeutic agents. For example, stent 10 can include one or more therapeutic agents that are disposed within pores 20 of generally tubular member 12. During delivery and/or use in a body of a subject, stent 10 can elute the therapeutic agents. For example, as generally tubular member 12 erodes, the therapeutic agents within pores 20 can be released into the body. The erosion of generally tubular member 12 can result in a relatively consistent release of therapeutic agent, as pores 20 continue to become exposed. Examples of therapeutic agents include non-genetic therapeutic agents, genetic therapeutic agents, vectors for delivery of genetic therapeutic agents, cells, and therapeutic agents identified as candidates for vascular treatment regimens, for example, as agents targeting restenosis. Therapeutic agents are described, for example, in Weber, U.S. Patent Application Publication No. US 2005/0261760 A1, published on Nov. 24, 2005, and entitled “Medical Devices and Methods of Making the Same”, and in Colen et al., U.S. Patent Application Publication No. US 2005/0192657 A1, published on Sep. 1, 2005, and entitled “Medical Devices”.

In certain embodiments, the sizes of pores 20 and/or arrangement of pores 20 in generally tubular member 12, and/or the volume percent of generally tubular member 12 that is occupied by pores 20, can be selected to achieve a desired pattern and/or rate of erosion of generally tubular member 12.

Generally, as pores 20 in a region of generally tubular member 12 become larger (as one or more of the dimensions of the pores increase), the erosion rate of that region can increase. In some embodiments, one or more of the pores in generally tubular member 12 can have a cross-sectional dimension (e.g., length, width, diameter) of at least about 20 nanometers (e.g., at least about 50 nanometers, at least about 100 nanometers, at least about 250 nanometers, at least about 500 nanometers, at least about 750 nanometers, at least about one micron, at least about five microns, at least about 10 microns, at least about 25 microns, at least about 40 microns, at least about 50 microns, at least about 75 microns) and/or at most about 100 microns (e.g., at most about 75 microns, at most about 50 microns, at most about 40 microns, at most about 25 microns, at most about 10 microns, at most about five microns, at most about one micron, at most about 750 nanometers, at most about 500 nanometers, at most about 250 nanometers, at most about 100 nanometers, at most about 50 nanometers). In certain embodiments, one or more of the pores in one region of generally tubular member 12 can have a cross-sectional dimension of from about 20 nanometers to about 10 microns, while one or more of the pores in another region of generally tubular member 12 can have a cross-sectional dimension of from about 10 microns to about 100 microns.

Typically, as the volume percent of a region of generally tubular member 12 that is occupied by pores 20 increases, the erosion rate of that region can also increase. Thus, if it is desirable for certain regions of generally tubular member 12 to erode more quickly than other regions of generally tubular member 12, the quickly eroding regions may be designed to have a higher volume percent that is occupied by pores 20 than the slowly eroding regions. In some embodiments, the pores in one or more regions (e.g., all) of generally tubular member 12 can occupy at least about five percent (e.g., at least about 10 percent, at least about 20 percent, at least about 30 percent, at least about 40 percent, at least about 50 percent, at least about 60 percent, at least about 70 percent, at least about 80 percent, at least about 90 percent), and/or at most about 95 percent (e.g., at most about 90 percent, at most about 80 percent, at most about 70 percent, at most about 60 percent, at most about 50 percent, at most about 40 percent, at most about 30 percent, at most about 20 percent, at most about 10 percent), of the volume of the bioerodible foam. In certain embodiments, the pores in one region of generally tubular member 12 can occupy from about five percent to about 50 percent of the volume of the region, while the pores in another region of generally tubular member 12 can occupy from about 50 percent to about 95 percent of the volume of the other region. As used herein, the volume percent of the pores in a sample of metal foam is calculated according to formula (1) below, in which D_{M} is the density of the bulk material of the metal foam, and D_{S} is the density of the sample of metal foam:

Volume Percent of Pores=[(D_{M}−D_{S})/D_{M}]×100% (1)

In some embodiments, pores 20 can be provided in an arrangement that can affect the erosion rate of generally tubular member 12. For example, in certain embodiments, one region of generally tubular member 12 can be designed to have a relatively high pore density, and/or to have pores 20 with relatively large cross-sectional dimensions, while another region of generally tubular member 12 can be designed to have a relatively low pore density, and/or to have pores 20 with relatively small cross-sectional dimensions. The region of generally tubular member 12 with the relatively high pore density and/or including pores 20 with relatively large cross-sectional dimensions may erode at a faster rate than the other region of generally tubular member 12.

In some embodiments, the dimensions of pores 20, density of pores 20, and/or arrangement of pores 20 can be selected to achieve a desired pattern and/or rate of elution of therapeutic agent from generally tubular member 12.

Typically, a region of generally tubular member 12 including pores 20 with relatively large cross-sectional dimensions can elute therapeutic agent at a faster rate than a region of generally tubular member 12 including pores 20 with relatively small cross-sectional dimensions.

Generally, a region of generally tubular member 12 including a relatively high density of pores 20 can elute therapeutic agent at a faster rate than a region of generally tubular member 12 including a relatively low density of pores 20.

In some embodiments, one region of generally tubular member 12 can be designed to have a relatively high pore density, and/or to have pores 20 with relatively large cross-sectional dimensions, while another region of generally tubular member 12 can be designed to have a relatively low pore density, and/or have pores 20 with relatively small cross-sectional dimensions. The region of generally tubular member 12 with the relatively high pore density, and/or including pores 20 with relatively large cross-sectional dimensions, may elute therapeutic agent at a faster rate than the other region of generally tubular member 12.

Generally tubular member 12 of stent 10 can be formed, for example, by cutting a tubular shape out of a metal foam block. In some embodiments, generally tubular member 12 can be formed by cutting a strip out of a metal foam block, rolling the strip, and welding its ends together to form generally tubular member 12. In certain embodiments, generally tubular member 12 can be formed by pouring liquid metal foam into a mold in the shape of generally tubular member 12. Liquid metal foam can be formed, for example, by melting a metal to form molten metal, and injecting gas (e.g., air) and/or one or more foaming agents into the molten metal. A foaming agent is a material that can decompose to release gas under certain conditions (e.g., elevated temperature). An example of a foaming agent that can be used to produce a metal foam is powdered titanium hydride, which can decompose to form titanium and hydrogen gas at elevated temperatures. In certain embodiments, generally tubular member 12 can be formed by molding a mixture of a bioerodible metal and a second bioerodible material into a generally tubular shape, and exposing the generally tubular shape to a solvent that solvates the second bioerodible material (without also solvating the bioerodible metal), and/or to a temperature that causes the second bioerodible material to melt (without also causing the bioerodible metal to melt). When the second bioerodible material is solvated and/or when it melts, it can result in the formation of pores in the metal, thereby producing a metal foam.

While a stent including a generally tubular member formed out of a metal foam and/or including a therapeutic agent has been described, in some embodiments, a stent can include one or more other materials. The other materials can be used, for example, to enhance the strength and/or structural support of the stent. Examples of other materials that can be used in conjunction with a metal foam in a stent include metals (e.g., titanium, tantalum, cobalt, chromium, niobium), metal alloys (e.g., 316L stainless steel, cobalt alloys such as HAYNES® alloy 25 (L605), Nitinol, niobium alloys such as NblZr, titanium alloys such as Ti6Al4V), and/or polymers (e.g., styrene-isobutylene styrene (SIBS)). As an example, in some embodiments, a stent can include a generally tubular member formed out of a porous magnesium foam, and the pores in the generally tubular member can be filled with iron compounded with a therapeutic agent. The iron can, for example, enhance the strength and/or structural support of the stent, while also regulating the release of the therapeutic agent from the stent. In certain embodiments, a stent can include magnesium buffered with lithium and/or one or more rare earth elements (e.g., neodymium, praseodymium).

Additional examples of polymers that can be used in conjunction with a metal foam in a stent include polycarboxylic acid; polyethylene oxide; polyphosphazenes; polyanhydrides (e.g., maleic anhydride polymers); poly(alpha-hydroxy acid)s, such as polylactic acid (PLA), polyglycolic acid (PGA), and copolymers and mixtures thereof (e.g., poly(L-lactic acid) (PLLA), poly(D,L-lactide), poly(lactic acid-co-glycolic acid), 50/50 (DL-lactide-co-glycolide)); stereopolymers of L- and D-lactic acid; poly(lactic acid)/poly(glycolic acid)/polyethyleneglycol copolymers; copolymers of polyurethane and poly(lactic acid); copolymers of α-amino acids; copolymers of α-amino acids and caproic acid; copolymers of α-benzyl glutamate and polyethylene glycol; copolymers of succinate and poly(glycols); polyphosphazene; polyhydroxy-alkanoates; copolymers of bis(p-carboxyphenoxy) propane acid and sebacic acid; sebacic acid copolymers; polyhydroxybutyrate and its copolymers; polypropylene fumarate; polydepsipeptides; polydioxanones; polyoxalates; poly(α-esters); polycaprolactones and copolymers and mixtures thereof (e.g., poly(D,L-lactide-co-caprolactone), polycaprolactone co-butylacrylate); polyhydroxybutyrate valerate and blends; polycarbonates (e.g., tyrosine-derived polycarbonates and acrylates, polyiminocarbonates, polydimethyltrimethyl-carbonates); polyglycosaminoglycans; macromolecules such as polysaccharides (e.g., hyaluronic acid, celluloses, hydroxypropylmethyl cellulose, gelatin, starches, dextrans, alginates, and derivates thereof); polypeptides; polygluconate; polylactic acid-polyethylene oxide copolymers; modified cellulose; poly(hydroxybutyrate); polyanhydrides (e.g., crystalline polyanhydrides, amorphous polyanhydrides); polyacetates; maleic anyhydride copolymers; polyorthoesters; polyphosphoester; poly-amino acids; polyamides; and mixtures and copolymers thereof. Typically, PGA and polydioxanone can erode relatively quickly (e.g., over a period of a few weeks to a few months), while PLA and polycaprolactone can erode relatively slowly (e.g., over a period of a few months to a few years).

Further example of materials that can be used in conjunction with a metal foam in a stent include proteins (e.g., collagen, fibrin, elastin); glycoproteins (e.g., vitronectin, fibronectin, laminin); cyanoacrylates; calcium phosphates (e.g., zinc-calcium phosphate); reconstituted basement membrane matrices; glycosaminoglycans; and derivatives and mixtures thereof.

In certain embodiments, a stent can include both a bioerodible metal foam and one or more other materials (e.g., starches, sugars) that are bioerodible. The metal foam and the other materials may erode at different rates. Thus, the other biocrodible materials can be added to the metal foam to, for example, tailor the erosion rate of the stent. For example, a stent may include a generally tubular member that is formed of a bioerodible metal foam. A bioerodible polymer may be disposed within some or all of the pores of the metal foam. Examples of bioerodible polymers include polyiminocarbonates, polycarbonates, polyarylates, polylactides, and polyglycolic esters. A stent including a metal foam and a bioerodible polymer disposed within the pores of the metal foam may be made, for example, by forming a generally tubular member out of a metal foam (e.g., as described above), immersing the generally tubular member in a solution of the polymer, and allowing the solution to dry, so that the solvent in the solution evaporates, and the polymer is left behind on the stent.

In some embodiments, a stent can include a bioerodible metal foam and one or more other materials that carry a therapeutic agent. For example, a stent may include a generally tubular member that is formed of a metal foam including pores. A polymer containing a therapeutic agent can be disposed within the pores. The polymer may be non-biocrodible, or may be bioerodible. In embodiments in which the polymer is bioerodible, the polymer may erode at a different rate from the metal foam. As an example, in some embodiments, the polymer can erode at a faster rate than the metal foam, causing all of the therapeutic agent to be released into the body before the generally tubular member has completely eroded. As another example, in certain embodiments, the polymer can erode at a slower rate than the metal foam. The result can be that after the foam has completely eroded, at least some of the therapeutic-agent containing polymer can remain in the body (e.g., in the form of polymeric particles). In some embodiments in which the stent has been delivered into a lumen of a subject, the polymer can be at least partially embedded in a wall of the lumen. As the polymer continues to erode, it can release the therapeutic agent into the body. Thus, the body can continue to be treated with the therapeutic agent, even after the generally tubular member has eroded. The therapeutic agent can be selected, for example, to alleviate the effects, if any, of the erosion of the stent on the body. By including a material (such as a polymer) containing a therapeutic agent, the stent can have a therapeutic agent elution rate that is independent of the erosion rate of its generally tubular member.

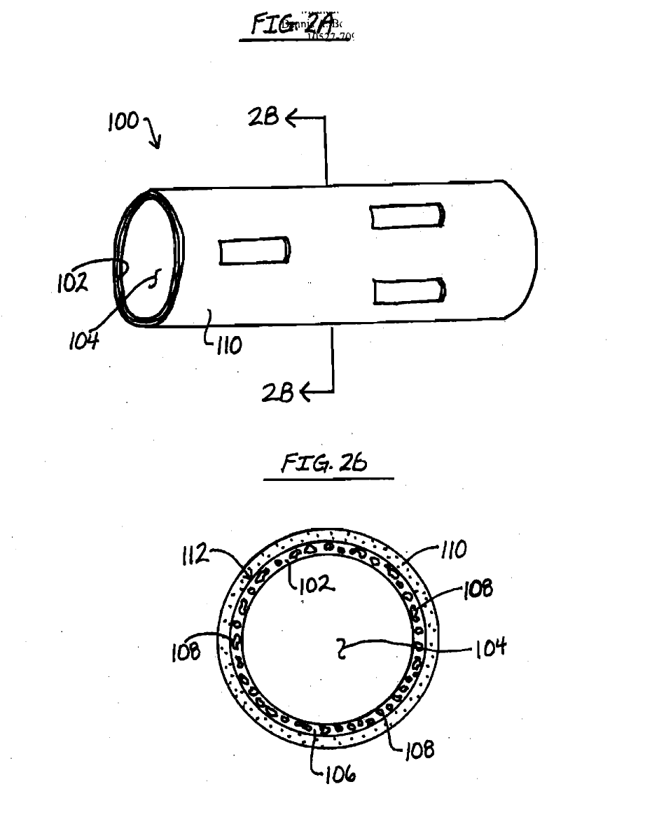

In certain embodiments, a stent can include one or more coatings on one or more surfaces of the stent. For example, FIGS. 2A and 2B show a stent 100 including a generally tubular member 102 defining a lumen 104. Generally tubular member 102 is formed of a metal foam 106 including pores 108. Stent 100 further includes a coating 110 disposed on the outer surface 112 of generally tubular member 102. Coating 110 can be used, for example, to regulate therapeutic agent release from generally tubular member 102. For example, pores 108 may contain one or more therapeutic agents, and coating 110 (e.g., which may be bioerodible) may be used to control the release of the therapeutic agents from pores 108 (e.g., by delaying the release of the therapeutic agents until stent 100 has reached a target site).

In certain embodiments, a stent can include a coating that contains a therapeutic agent or that is formed of a therapeutic agent. For example, a stent may include a coating that is formed of a polymer and a therapeutic agent. The coating can be applied to a generally tubular member of the stent by, for example, dip-coating the generally tubular member in a solution including the polymer and the therapeutic agent. In some embodiments, a vacuum-loading process can be used to load a therapeutic agent onto a stent. For example, a porous stent can be placed in a vacuum chamber, and a vacuum can be applied to remove air from the pores. Thereafter, a coating (e.g., formed of a therapeutic agent) can be added onto the stent so that the coating fills the pores, and then the vacuum can be removed. In certain embodiments, a pressure filling process can be used to load a therapeutic agent onto a stent. The pressure filling process can be used, for example, to displace the air in the pores in a porous stent, and fill the pores with a therapeutic agent. For example, in some embodiments, a tube with holes or relatively large pores in it can be placed within a lumen of a stent. Then, a coating solution can be pressure fed through the tube and out the holes or pores of the tube, so that the coating solution flows into the pores of the stent. The result can be that a pressure differential is established between the inner diameter of the stent to the outer diameter of the stent, such that the coating solution is driven into the pores of the stent.

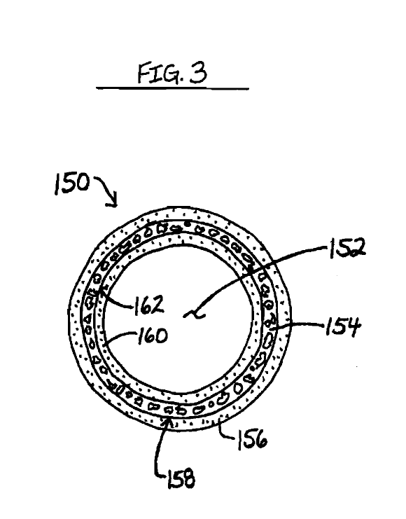

While a stent with one coating has been shown, in some embodiments, a stent can include multiple (e.g., two, three, four, five) coatings. For example, FIG. 3 shows a cross-sectional view of a stent 150 having a lumen 152. Stent 150 includes a generally tubular member 154, and has a coating 156 on the outer surface 158 of generally tubular member 154, and a coating 160 on the inner surface 162 of generally tubular member 154. Coatings 156 and 160 can include one or more of the same materials, or can be formed of different materials.

Examples of coating materials that can be used on a stent include metals, metal oxides (e.g., iridium dioxide, zirconium oxide, titanium oxide), ceramics, and/or polymers. Ceramics are described, for example, in Shaw, U.S. Patent Application Publication No. US 2005/0163954 A1, published on Jul. 28, 2005, and entitled “Medical Devices”.

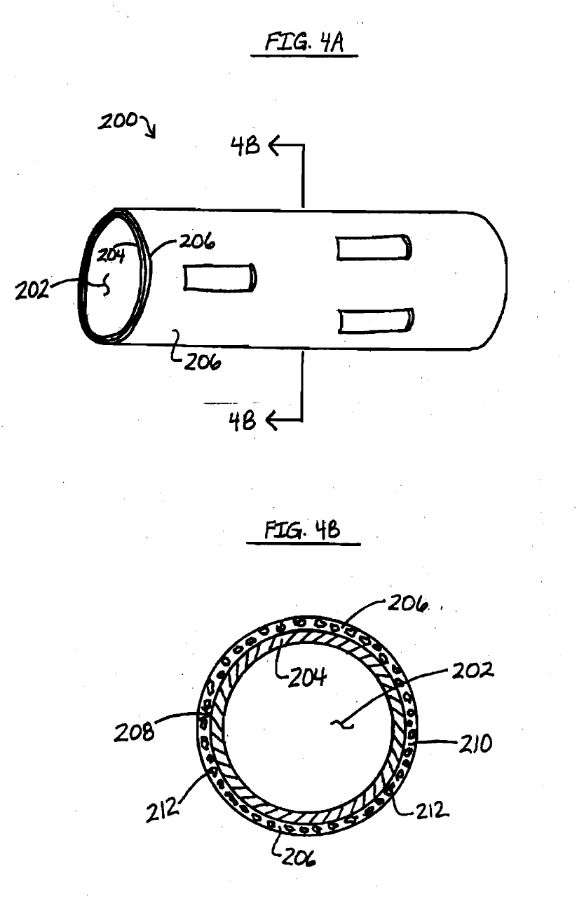

While stents including generally tubular members formed of a bioerodible metal foam have been described, in certain embodiments, a stent can alternatively or additionally include a coating that is formed of a bioerodible metal foam. For example, FIGS. 4A and 4B show a stent 200 having a lumen 202. Stent 200 includes a generally tubular member 204 that is not formed of a metal foam. Generally tubular member 204 may be formed of, for example, one or more metals (e.g., titanium, tantalum, cobalt, chromium, niobium), metal alloys (e.g., 316L stainless steel, cobalt alloys such as HAYNES® alloy 25 (L605), Nitinol, niobium alloys such as NblZr, titanium alloys such as Ti6Al4V), polymers, and/or other materials. Examples of polymers and other materials that can be used in generally tubular member 204 include the polymers and other materials described above as being suitable for use in conjunction with a metal foam. Stent 200 further includes a coating 206 that is disposed on the outer surface 208 of generally tubular member 204. Coating 206 is formed of a bioerodible metal foam 210 that includes pores 212. Metal foam 210 can be used, for example, as a reservoir for one or more therapeutic agents. For example, one or more therapeutic agents can be disposed within pores 212 of metal foam 210. During and/or after delivery of stent 200 to a target site in a body of a subject, metal foam 210 can erode, thereby eluting therapeutic agent into the body of the subject.

Coatings can be applied to a stent using, for example, dip-coating and/or spraying processes. As an example, in some embodiments, coating 206 can be applied to generally tubular member 204 by forming a liquid foam in which small gas bubbles are finely dispersed, and dipping generally tubular member 204 into the liquid foam. Alternatively or additionally, generally tubular member 204 can be sprayed with the liquid foam.

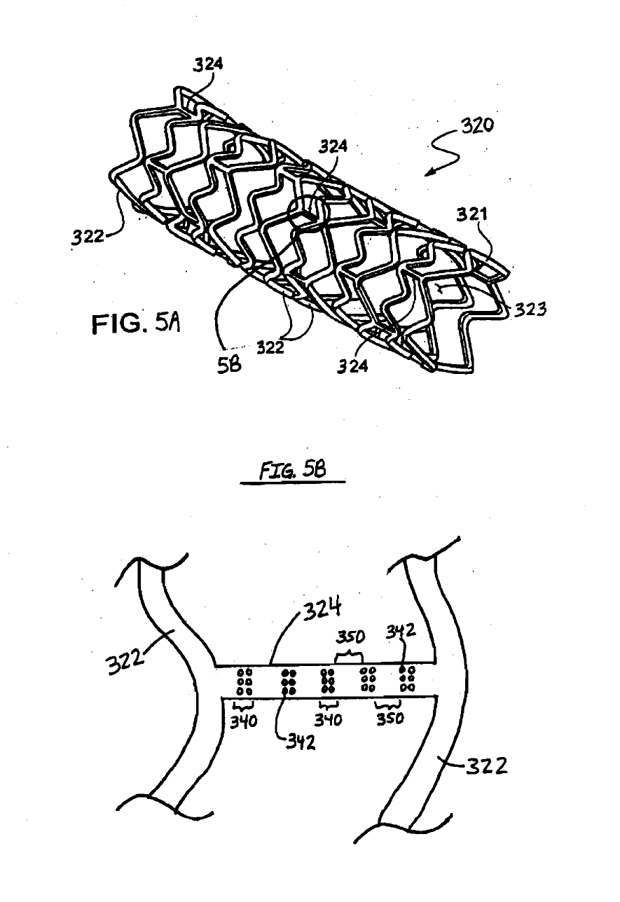

While a stent including a bioerodible metal foam has been described, in some embodiments, a stent can alternatively or additionally include one or more bioerodible metals that are not in the form of a foam. For example, FIG. 5A shows a stent 320 that is in the form of a generally tubular member 321 formed of a bioerodible metal. Generally tubular member 321 is defined by a plurality of bands 322 and a plurality of connectors 324 that extend between and connect adjacent bands. Generally tubular member 321 has a lumen 323. FIG. 5B shows a connector 324, which includes regions 340 including holes 342, and regions 350 that do not include any holes. During delivery and/or use of stent 320, bands 322 and/or connectors 324 can erode. The presence of holes 342 in regions 340 of connectors 324 can help to accelerate and/or control the erosion of connectors 324. The presence of holes 342 in regions 340 of connectors 324 may result in connectors 324 eroding at a faster rate than bands 322. In some embodiments, it may be desirable for connectors 324 to completely erode before bands 322, allowing stent 320 to move and flex within a target site (e.g., a lumen in a body of a subject). By the time connectors 324 have completely eroded, tissue may have grown over the remaining parts of stent 320 (e.g., bands 322), thereby helping to hold bands 322 (and, therefore, stent 320) in place.

Holes 342 can be formed, for example, using mechanical drilling and/or laser perforation techniques, and/or by applying water jets to regions 340 of connectors 324.

While regions 340 are shown as being uniformly spaced apart from each other, in some embodiments, a stent can include regions that have holes and that are not uniformly spaced apart from each other. Furthermore, while connector 324 in FIG. 5B is shown as having five regions 340 including holes 342, a component of a stent, such as a band or a connector, can have fewer regions including holes (e.g., three regions, one region), or can have more regions including holes (e.g., seven regions, 10 regions).

While a stent including connectors with regions including holes has been described, in some embodiments, another component of a stent can include one or more regions including holes. As an example, a stent may include both bands with regions including holes and connectors with regions including holes. In some embodiments, a stent can include a metal foam (e.g., a bioerodible metal foam), as well as one or more regions including holes. In certain embodiments, a stent can include a metal foam that has holes in it.

While certain embodiments have been described, other embodiments are possible.

As an example, in some embodiments, a stent including a generally tubular member formed of a bioerodible metal can be manufactured using powder metallurgy methods. For example, a stent can be formed by sintering and compacting bioerodible metal particles (e.g., in the form of a metal powder) into the shape of a generally tubular member. A metal particle can have a dimension of, for example, at least about 20 nanometers (e.g., at least about 50 nanometers, at least about 100 nanometers, at least about 250 nanometers, at least about 500 nanometers, at least about 750 nanometers, at least about one micron, at least about five microns, at least about 10 microns, at least about 25 microns, at least about 40 microns, at least about 50 microns, at least about 75 microns) and/or at most about 100 microns (e.g., at most about 75 microns, at most about 50 microns, at most about 40 microns, at most about 25 microns, at most about 10 microns, at most about five microns, at most about one micron, at most about 750 nanometers, at most about 500 nanometers, at most about 250 nanometers, at most about 100 nanometers, at most about 50 nanometers). Sintering the metal particles can include exposing the metal particles to a temperature of at least about 400° C. (e.g., at least about 500° C., at least about 750° C., at least about 1000° C.) and/or at most about 1550° C. (e.g., at most about 1000° C., at most about 750° C., at most about 500° C.). A generally tubular member that is formed by a sintering process may be porous or non-porous, or may include both porous regions and non-porous regions. In some embodiments in which the generally tubular member includes pores, the sizes of the pores can be controlled by the length of the sintering and compacting period, and/or by the temperature of the sintering process. In certain embodiments, a metal stent that is formed by sintering metal particles can erode after being used at a target site in a body of a subject, and the erosion of the metal stent can result in the formation of metal particles having the same size as the particles that were originally sintered together to form the stent. Thus, the size of the particles formed from the erosion of a stent can be selected, for example, by sintering metal particles of the desired size to form the stent. In some embodiments, a stent can be formed by sintering hollow metal particles into the shape of a generally tubular member. In certain embodiments, the resulting generally tubular member can be relatively light. Hollow metal particles can be formed, for example, by gas atomization of metal powders.

As another example, in some embodiments, a stent including a generally tubular member formed of a bioerodible metal can be manufactured using investment casting methods. For example, a generally tubular member can be cast in a pre-form. In certain embodiments, the pre-form can be water-soluble, and after the generally tubular member has been cast in the pre-form, the pre-form can be dissolved by contacting the pre-form with water. For example, in some embodiments, a mold of a generally tubular member can be filed with grains of sodium chloride. The sodium chloride grains can then be sintered in a furnace, such that the grains are fused together. Thereafter, a billet of metal can be placed on the sintered sodium chloride grains, and the assembly can be heated under vacuum to melt the metal. Once the metal has melted, an inert gas (e.g., argon) at high pressure can be used to force the molten metal into the spaces between the sintered sodium chloride grains. The sodium chloride can then be dissolved, thereby resulting in an open-cell metal foam.

As an additional example, in some embodiments, a stent including a generally tubular member formed of a bioerodible metal can be formed by deposition of the metal onto a pre-form. In certain embodiments, after the generally tubular member has been formed, the pre-form can be dissolved and/or melted to remove it from the generally tubular member. In some embodiments, an electrodeposition process can be used to form a generally tubular member of a stent. For example, a generally tubular member formed of an open-cell polyurethane foam can be made to conduct (e.g., by immersing the generally tubular member in a colloidal fluid dispersion of carbon black, and/or by vaporizing a thin layer of metal onto the generally tubular member). The generally tubular member can then be electroplated with metal and sintered to remove the polymer, resulting in a generally tubular member formed of an open cell metal foam.

As a further example, in some embodiments, a stent can include a generally tubular member including a syntactic metal foam. A syntactic metal foam can be formed, for example, by incorporating hollow spheres (e.g., hollow metal spheres and/or hollow ceramic spheres, such as hollow alumina spheres) into a molten metal. The resulting foam structure retains the hollow spheres. In certain embodiments, a syntactic metal foam can be relatively light.

As an additional example, while stents have been described, in some embodiments, other medical devices can include one or more foams, porous regions, holes, and/or biocrodible metals. For example, other types of endoprostheses, such as grafts and/or stent-grafts, may include one or more of the features of the stents described above. Additional examples of medical devices that may have one or more of these features include spinal implants, hip implants, artificial bones, and fixation hardware (e.g., screws, pins). In some embodiments in which a medical device includes one or more metal foams, the medical device can be relatively light, while also being relatively strong. In certain embodiments, bone that is in contact with a medical device including one or more metal foams can grow around the medical device and/or can adhere relatively well to the medical device.

As another example, while medical devices including open-cell metal foams have been described, in some embodiments, a medical device can alternatively or additionally include a closed-cell metal foam. Closed-cell metal foams include sealed pores that do not form an interconnected network. Closed-cell metal foams can be formed, for example, by injecting one or more gasses and/or foaming agents into molten metal. In certain embodiments, a medical device that includes (e.g., is formed of) one or more closed-cell metal foams can have relatively high structural integrity and/or strength, and/or can have a relatively low erosion rate (e.g., as compared to a medical devices that is formed of one or more open-cell metal foams).

As an additional example, in certain embodiments, a medical device can include one or more metal foams that are substantially non-bioerodible. In some embodiments, a medical device can include one or more Nitinol foams.

As a further example, in some embodiments, a vacuum molding process can be used to form a medical device, such as a stent. For example, a vacuum molding process can include using a vacuum to fill a mold of a stent with one or more biocrodible metals.

As another example, in some embodiments, a medical device can include regions that are formed of a metal foam (e.g., a biocrodible metal foam), and regions that are not formed of a metal foam. For example, a stent may include regions that are formed of a biocrodible metal foam, and regions that are formed of a metal that is neither bioerodible, nor in the form of a foam.

As an additional example, in certain embodiments, a medical device (e.g., a stent) including a metal foam coating may be further coated with one or more other coatings. The other coatings may be metal foams, or may not be metal foams.

As a further example, in some embodiments, a coating can be applied to certain regions of a medical device, while not being applied to other regions of the medical device.

As another example, in certain embodiments, a porous coating can be applied to a medical device (e.g., a stent) using a sintering process. For example, a porous coating may be applied to a stent by placing (e.g., electrostatically attaching) microspheres (e.g., polystyrene microspheres) onto a surface of the stent. A ceramic or metal oxide coating can then be coated over the microspheres (e.g., using a physical vapor deposition process). The stent can then be heated (e.g., to a temperature of at least about 190° C.), so that the microspheres melt and leave a porous structure behind.

As an additional example, in some embodiments, a medical device can include one or more bioerodible portions that are adapted to erode by a bulk erosion process, and one or more biocrodible portions that are adapted to erode by a surface erosion process. For example, a junction between one or more bands and/or connectors in a stent may be adapted to erode by a bulk erosion process, while the bands and/or connectors in the stent may be adapted to erode by a surface erosion process. The junction may erode at a faster rate than the bands and/or connectors which may, for example, result in enhanced longitudinal flexibility by the stent.

All publications, applications, references, and patents referred to in this application are herein incorporated by reference in their entirety.

Other embodiments are within the claims.

What is claimed is:

1. An endoprosthesis, comprising a generally tubular member comprising a biocrodible foam including a first metal.

2. The endoprosthesis of claim 1, wherein the generally tubular member further comprises a material that is non-bioerodible.

3. The endoprosthesis of claim 1, wherein the generally tubular member further comprises a second metal.

4. The endoprosthesis of claim 3, wherein the bioerodible foam includes pores, and the second metal is disposed within the pores.

5. The endoprosthesis of claim 1, wherein the generally tubular member further comprises a polymer.

6. The endoprosthesis of claim 5, wherein the polymer is biocrodible.

7. The endoprosthesis of claim 5, wherein the bioerodible foam includes pores, and the polymer is disposed within the pores.

8. The endoprosthesis of claim 1, wherein the first metal is selected from the group consisting of iron, magnesium, zinc, aluminum, and combinations thereof.

9. The endoprosthesis of claim 1, wherein the first metal comprises iron.

10. The endoprosthesis of claim 1, wherein the first metal comprises magnesium.

11. The endoprosthesis of claim 1, wherein the bioerodible foam has a volume and the bioerodible foam includes pores that occupy at least about five percent of the volume of the bioerodible foam.

12. The endoprosthesis of claim 1, wherein the bioerodible foam has a volume and the bioerodible foam includes pores that occupy at most about 95 percent of the volume of the bioerodible foam.

13. The endoprosthesis of claim 1, further comprising a therapeutic agent.

14. An endoprosthesis, comprising:
a generally tubular member comprising a bioerodible metal and having a first region including a least one hole and a second region that does not include any holes,
wherein both the first region and the second region include the bioerodible metal.

15. The endoprosthesis of claim 14, wherein the generally tubular member comprises a connector including at least one of the first region and the second region.

16. The endoprosthesis of claim 14,wherein the generally tubular member comprises a band including at least one of the first region and the second region.

17. The endoprosthesis of claim 14, wherein the bioerodible metal is selected from the group consisting of iron, magnesium, zinc, aluminum, and combinations thereof.

18. A method of making an endoprosthesis comprising a generally tubular member, the method comprising:
heating a powder comprising a bioerodible metal to form the generally tubular member.

19. The method of claim 18, wherein the biocrodible metal is selected from the group consisting of iron, magnesium, zinc, aluminum, and combinations thereof.

20. A method of making an endoprosthesis comprising a generally tubular member, the method comprising:
treating a bioerodible foam comprising a first metal to form the generally tubular member.

21. The method of claim 20, wherein treating a bioerodible foam comprising a first metal to form the generally tubular member comprises molding the biocrodible foam to form the generally tubular member.

22. The method of claim 20, wherein the generally tubular member includes a generally tubular portion, and treating a bioerodible foam comprising a first metal to form the generally tubular member comprises coating the generally tubular portion with the biocrodible foam.

23. The method of claim 20, wherein the first metal is selected from the group consisting of iron, magnesium, zinc, aluminum, and combinations thereof.

24. The method of claim 20, further comprising combining the biocrodible foam with a second metal.

25. The method of claim 24, wherein the bioerodible foam includes pores, and combining the foam with a second metal comprises infiltrating the pores with the second metal.

26. The method of claim 20, further comprising combining the bioerodible foam with a polymer.

27. The method of claim 26, wherein the bioerodible foam includes pores, and combining the biocrodible foam with a polymer comprises infiltrating the pores with the polymer.

28. The method of claim 20, further comprising adding a therapeutic agent to the generally tubular member.

29. A method of making an endoprosthesis comprising a generally tubular member, the method comprising:
forming at least one hole in a first region of the generally tubular member so that the first region includes the at least one hole and a second region of the generally tubular member does not include any holes,
wherein the first region of the generally tubular member includes a biocrodible metal.

30. The method of claim 29, wherein the second region includes the bioerodible metal.

31. The method of claim 29, wherein the bioerodible metal is selected from the group consisting of iron, magnesium, zinc, aluminum, and combinations thereof.
